(19) United States
(12) Patent Application Publication
(10) Pub. No.: US 2019/0111178 A1
(43) Pub. Date: Apr. 18, 2019

(54) METHODS AND COMPOSITIONS FOR THE TREATMENT OF INTERVERTEBRAL DISC HERNIATION

(71) Applicant: GU Ventures AB, Gothenburg (SE)

(72) Inventors: Kjell Olmarker, Molndal (SE); Daniel Jonsson, Goteborg (SE)

(21) Appl. No.: 16/090,936

(22) PCT Filed: Apr. 2, 2017
(86) PCT No.: PCT/SE2017/050327

§ 371 Date: Oct. 3, 2018

(30) Foreign Application Priority Data
Apr. 4, 2016 (SE) 1650449-0

(51) Int. Cl. A61L 27/36; A61K 38/17; A61P 19/04; C07K 16/28; C07K 14/705; A61K 47/42; A61K 47/68

(52) U.S. Cl. CPC A61L 27/3658; A61K 38/1774; A61P 19/04; A61K 47/68; C07K 16/283; C07K 14/70521; A61K 47/42; C07K 16/2818

(57) ABSTRACT

The invention relates to methods and compositions for use in the treatment or prophylaxis of intervertebral disc herniation in a mammal. The composition according to the invention comprises an inhibitor of T-cell activation, said inhibitor being capable of inhibiting CD28-mediated co-stimulation of T-cells. The said inhibitor of T-cell activation is preferably a protein comprising either an exact or a modified version of the extracellular domain of CTLA-4, such as abatacept, belatacept, XPro9523 and/or ASP2408.

### TECHNICAL FIELD

The invention relates to methods and compositions for use in the treatment or prophylaxis of intervertebral disc herniation in a mammal. The composition according to the invention comprises an inhibitor of T-cell activation, said inhibitor being capable of inhibiting CD28-mediated co-stimulation of T-cells. The said inhibitor of T-cell activation is preferably a protein comprising either an exact or a modified version of the extracellular domain of CTLA-4, such as abatacept, belatacept, XPro9523 and/or ASP2408.

### BACKGROUND ART

The human spinal column is made up by a number of vertebrae stacked on top of each other. Two adjacent vertebrae are connected via two intervertebral joints (or facet joints) and the intervertebral disc, which together constitute a motion segment allowing for flexion/extension, lateral flexion and rotation of the spine. The spine is further stabilized by numerous ligaments and muscles.

The intervertebral disc consists of an outer, fibrous structure called annulus fibrosus, an inner gel-like core called nucleus pulposus and two cartilaginous endplates connecting the disc to the two adjacent vertebrae. The nucleus pulposus has a high water content crucial for its biomechanical properties in terms of allowing movement and absorbing axial load. In ageing, the water concentration commonly decreases, which is a part of a phenomenon called disc degeneration. Other signs of disc degeneration include a decrease in disc height and deterioration of the annulus fibrosus [1].

In disc herniation, the deterioration of the annulus fibrosus causes it to rupture, not seldom due to excessive mechanical strain during e.g. heavy lifting. The rupture allows for the displacement of nucleus pulposus dorsally into the spinal canal or the intervertebral foramen. This can cause pressure on an adjacent nerve root which, in combination with chemical factors as further discussed below, causes radiating pain. The hernia is most commonly located in the lower lumbar spine and symptoms typically manifest in form of low back pain closely followed by radiating pain. The overall incidence is 1-2%, and it is most common in middle-aged men and women, though the age-span stretches from teenagers to the elderly. It is also worth mentioning that asymptomatic hernias are highly common in middle-aged, healthy volunteers. Typically, the symptoms from a disc herniation improve spontaneously within 2-4 weeks after onset and the hernia eventually resorbs. However, for about 10% of patients the pain becomes chronic. In these cases, surgery is the preferred treatment option today, in which the disc fragment causing pressure on the nerve is removed.

For a long time, sciatic pain in disc herniation was believed to be caused solely by mechanical pressure on a nerve root, but during the last three decades this has been proven wrong. Studies have shown that pressure alone can cause paresthesia, dysesthesia or weakness, but not pain [2], and numerous experimental studies as well as clinical observations have indicated that chemical factors are required for the onset of pain [3]. Also, autologous nucleus pulposus placed on a nerve root, without pressure, has been found to induce both structural and neurophysiological damages [4-6]. One of the specific chemical factors first discovered to play a major role in the induction of these damages was the pro-inflammatory cytokine TNFα [7]. Various studies have indicated TNFα as a key factor in the pathophysiology of disc herniation and degeneration, and there is also evidence that several other pro-inflammatory cytokines, such as IL-1 and IL-6, are important as well [8, 9]. Clinical trials have been performed using TNF-inhibitors for sciatica, with contradictory results [10, 11].

Though increased levels of pro-inflammatory cytokines are recognized to be important in disc herniation, it is still not clear what causes this increase. Experimental studies have detected pro-inflammatory cytokines in intervertebral discs [12], which led to the hypothesis that the cytokines present in herniated disc material were cytokines produced by the cells of the displaced disc material. Others have found that the levels of cytokines increase after displacement into the spinal canal [13], suggesting that there is an ongoing inflammatory reaction following herniation that causes the increase of cytokines. This hypothesis is supported by several other studies [14, 15].

Some studies have demonstrated the involvement of the adaptive immune system in disc herniation, suggesting an autoimmune reaction against nucleus pulposus. This hypothesis is supported by the avascular nature of the healthy nucleus pulposus, which is thus not exposed to the immune system as long as it is contained in the center of the disc. In 1988, Pennington et al. identified IgG in the nucleus pulposus in healthy canine intervertebral discs [16]. Later studies have detected autoantibodies both in herniated disc material and in degenerated discs of humans [17, 18], and experimental studies have shown that exposure to nucleus pulposus can cause activation of T-cells [19]. A recent study also found elevated serum levels of the pro-inflammatory cytokines IL-6 and IL-8 in patients with disc herniation, thus suggesting a systemic inflammatory reaction. A correlation was also found between high serum levels of these cytokines and severity of symptoms in inclusion and poor long-term outcome [20]. Consequently, it is recognized that an inflammatory reaction occurs in disc herniation that may be caused by an autoimmune reaction to the nucleus pulposus, but the extent and importance of such immunologic response are not fully understood. The potential involvement of T-cells in the pathophysiology of disc herniation has only been briefly suggested, and no attempts have been made to define their role in terms of, e.g., importance for cytokine production, importance in the morphological development of disc hernias, or correlation with clinical severity of symptoms.

Histological analyses of human disc hernias have found that the hernias not only comprise components of the disc, such as nucleus pulposus (NP) and annulus fibrosus, but also granulation tissue [21-23]. The granulation tissue commonly surrounds the herniated NP and is also commonly infiltrated by macrophages, suggesting an inflammatory reaction surrounding the NP. Other common histological and immunohistochemical findings in these studies were neovascularization and expression of TNF, matrix metalloproteinase 3, basic fibroblast growth factor and vascular endothelial growth factor. Most of these findings were particularly abundant in extruded and sequestrated hernias. These types of hernia also commonly have a larger amount of granulation tissue than bulges and protrusions [24]. It has also been observed that granulation tissue is more common in younger patients [24, 25]. However, these different histological characteristics have not been found useful in predicting clinical outcome [26].

Several follow-up assessments of the progression of the size of lumbar disc hernias have been published. The results from these suggest a more dynamic progress than what was previously thought for herniated discs. For example, Jensen et al observed spontaneous regression in the vast majority (75-100%) of broad-based protrusions, extrusions and sequestrations, but less so in focal protrusions (35%) and bulges (3%) in a prospective 14-month follow-up study of a symptomatic cohort (n=154) [27]. Takida et al did follow-up MRI every 3 months in a symptomatic cohort and found a similarly high regression rate for extrusions and sequestrations, but less so for protrusions [28]. They also found a correlation between morphologic regression and a favorable clinical outcome. Other studies have failed to correlate morphological development with clinical outcome, and as such this correlation remains a controversial subject. Other imaging characteristics predictive of spontaneous regression include contrast enhancement in the hernia tissue [29] and high signal intensity on T2-weighted sequences [30].

Based on the data discussed above it has been stipulated that there is a correlation between spontaneous regression in disc hernias and the inflammation in the hernia. This has been recognized, discussed and also partly investigated in the literature for several decades. In 1998 Matsui et al. suggested that MMP-1 and MMP-3 cause degradation of the herniated disc extracellular matrix and that this was associated with the inflammatory reaction in the hernia [23]. In 2009 Genevay et al. published an article supporting this hypothesis while also suggesting that inhibition of the inflammatory mechanisms causing the increase in MMP-1 and MMP-3, particularly TNF, could inhibit the spontaneous resorption of disc hernias [31]. In 2004 Kato et al. published data further supporting this hypothesis in 2004, and they also proposed a reaction cascade leading to spontaneous resorption of disc hernias [31] (FIG. 4). They suggest inflammation induced by an interaction between disc cartilage and macrophages creates an inflammatory environment, with increase of TNF causing increased vascularization through increased expression of vascular endothelial growth factor which both allows more macrophages to access the herniated tissue and thus increase inflammation. TNF then activates and increases the expression of MMP's, leading to degradation of the extracellular matrix and thus spontaneous resorption of the hernia. This hypothesis, that inflammation is the mechanism that causes regression of the disc hernia, has been repeatedly stated in more recent literature as well, both in original papers [32] and in reviews [33]. Given the above mentioned hypothesis regarding autoimmunity as a driver of inflammation in disc herniation, it is reasonable to consider that this autoimmune reaction contributes to regression of the hernia.

Experimental disc puncture in the caudal spine has become a widely accepted model for disc degeneration in the rat [34-37], but not yet for herniation. However, it has been shown that disc puncture in the lumbar spine causes the development of a hernia-like nodule on the surface of the disc [38], as noted with macroscopic analysis three weeks after puncture. A histological analysis showed that the nodule consisted mainly of granulation tissue, which also can be found in human disc herniations. Interestingly, discography, a certain MRI investigation in clinical use today, which involves inserting a needle into a disc, has been found to cause increased prevalence of degeneration and herniations in follow-up assessments of patients undergoing the procedure [39], suggesting that “disc puncture” in humans can cause herniation. The formation of the nodule was shown to be caused by the presence of nucleus pulposus on the surface of the disc, not the disc damage itself. Consequently, a disc herniation could form as the result of an autoimmune reaction to a relatively small amount of nucleus pulposus in the spinal canal, rather than the widely accepted concept that nucleus pulposus simply dorsally displaces when the annulus fibrosus ruptures.

In one study [40], two common anti-rheumatic drugs, infliximab (a selective TNF-inhibitor) and methotrexate (an anti-inflammatory drug with a wide mechanism of action), were administered to rats following disc puncture. Neither of the drugs administered showed any effect on the formation of nucleus pulposus-induced disc hernia-like nodules on the disc surface, indicating that TNF is not essential for the morphological formation of disc hernias.

The T-cell is a critical component of the adaptive immune system, and its activation is a crucial first step in the activation of both cellular and humoral immune responses. A productive T-cell activation requires not only the presentation of an antigen by an antigen presenting cell (APC) that binds to the T-cell receptor (TCR), but also co-ligation of other surface receptors and ligands between the APC and the T-cell. This process is commonly referred to as co-stimulation. In the absence of co-stimulatory signaling, the T-cell remains unresponsive (anergy) regardless of TCR affinity to the antigen [41].

One of the most recognized co-stimulatory signals is the interaction between CD28, CD80 and CD86 [41, 42]. CD80/86 are expressed on the surface of APCs and CD28 is expressed on the surface of T-cells. CD80/86 binds to CD28, causing a strong co-stimulatory signal which thus allows for T-cell activation. Delicate regulation of CD80/86 and CD28 expression is required to allow for both effective cellular and humoral responses to microorganisms while still maintaining tolerance to self and preventing autoimmunity. Another mechanism through which this co-stimulatory signal is regulated is through the expression of Cytotoxic T lymphocyte antigen-4 (CTLA-4). CTLA-4 is expressed on the surface of the T-cell and also binds to CD80/86, but in contrast to that of CD28-CD80/86, the interaction between CTLA-4-CD80/86 limits the expansion and proliferation of the T-cell. Expression of CTLA-4 may thus be seen as a mechanism through which T-cells can limit their own activation, particularly important for maintaining self-tolerance.

Fusion proteins comprising the extracellular domain of CTLA-4 are known in the art, e.g. from WO 93/000431 and WO 01/92337, and have been developed under the names abatacept and belatacept, which are dimers comprising two monomers shown as SEQ ID NO: 1 and 2, respectively. They bind to CD80/86, thus preventing the co-stimulatory signal required for T-cell activation. Abatacept (trade name Orencia®) and belatacept (trade name Nulojix®) have been described for the use against autoimmune diseases such as rheumatoid arthritis (U.S. Pat. No. 7,455,835); juvenile rheumatoid arthritis (U.S. Pat. No. 8,703,718); type I diabetes mellitus (U.S. Pat. No. 8,497,247); Sjögren's syndrome (U.S. Pat. No. 8,722,632); and against transplant rejection (U.S. Pat. No. 7,439,230).

Modified versions of these drugs that allow for higher affinity to CD80/86, different affinity profile to CD80/86, and/or prolonged half-life, are also known in the art. These attributes may allow for better efficacy, lower and/or less frequent dosages and fewer side effects. Such drugs include, but are not limited to, ASP2408 [43-45] and XPro9523 [46]. Other known examples of such compounds include ASP2409, Xtend-CTLA4, M834, as well as mutants from wild-type CTLA4-Ig. The said variants are disclosed in e.g. U.S. Pat. Nos. 8,883,971, 8,642,557, 8,629,113, 8,496,935, 8,491,899, 8,445,230, 8,329,867, 8,318,176, 8,283,447, 8,268,587, 8,071,095, 7,794,718, WO 2011/113019, WO 2009/058564, EP 2863936, EP 2855533, EP 2612868, EP 2612867, EP 2385065, WO 2011/113019, and WO 2009/058564.

Another mechanism through which CD28-mediated co-stimulation of T-cells can be inhibited is through administration of an antagonist anti-CD28 compound, including but not limited to, FR104 as disclosed in WO 2011/101791 [47-49].

WO 00/75659 A1, “Antibodies to nucleus pulposus in disc herniation, diagnostic kit, medical preparations and treatment” concerns methods for treating disc herniation through various hypothetical ways of neutralizing the effects of serum antibodies towards nucleus pulposus cells. The inventors do not disclose experimental data supporting efficacy of any of the suggested ways of neutralizing nucleus pulposus antibodies in the treatment of disc herniation. Also, importantly, the publication discloses preventing the effects of a humoral response to nucleus pulposus. This differs from the current invention which specifically targets cellular immunity (i.e. T-cells through inhibition of CD28-mediated co-stimulation of T-cells). Furthermore, the inventors make no reference to potential treatment effects on the morphology of the hernias.

WO 2015/070840 A2, “Stimulating bone formation by inhibition of cd28 co-stimulation” concerns a method for stimulating bone formation by application of an inhibitor of CD28-mediated co-stimulation of T-cells. In one embodiment, the invention relates to various ways of improving bone grafting by either systemic or local application of a molecule providing inhibition of CD28-mediated co-stimulation. Furthermore, bone grafting is discussed in the context of performing spinal fusion. Disc herniation is mentioned as a possible indication for spinal fusion. Spinal fusion is a type of surgical procedure which aims to join two or more vertebrae with the purpose of preventing any movement between the fused vertebrae, commonly to reduce motion-related segmental back pain. It involves bone grafting, i.e. bridging of the vertebrae with bone grafts from the patient, a donor or artificial bone substitutes. The bone graft is commonly placed over the facet joints and/or in the intervertebral space after evacuation of the intervertebral disc. Following surgery, new bone forms through the bone graft creating a bony connection between the fused vertebrae, thus eliminating movement in the fused segment/-s.

Importantly, spinal fusion alone is not a treatment for disc herniation. It is occasionally offered as a supplement to conventional disc herniation surgery for patients with a high risk of recurrence and/or pronounced back pain. Furthermore, the inventors do not claim or discuss any potential efficacy of inhibition of CD28-mediated co-stimulation on the pathophysiology of disc herniation, nor any effects on disc tissue at all. They describe a method of placing bone graft in the area usually occupied by the intervertebral disc, with the purpose of achieving fusion between the vertebral endplates. This is not to be understood as having any effects on disc tissue, but as they also mention the standard procedure of such fusion involves evacuation of the intervertebral disc space (removal of the intervertebral disc).

### BRIEF DESCRIPTION OF THE DRAWINGS

FIG. 1: Nodule size as determined by objective measurements of MRI images for the abatacept study. The chart displays mean values with error bars representing+1 SD. Asterisks mark differences of statistical significance (*=p≤0.05, **=p≤0.01).

FIG. 2: Nodule size as determined by subjective estimation of MRI images for the abatacept study. The chart displays mean values with error bars representing+1 SD. Asterisks mark differences of statistical significance (*=p≤0.05, **=p≤0.01).

FIG. 3: Nodule size as determined by macroscopic analysis after euthanization and dissection upon termination of the study (week 8), abatacept study. 0=no nodule, 1=small nodule, 2=clear nodule, 3=pronounced nodule. The chart displays mean values with error bars representing+1 SD. Significant differences are indicated with asterisks (*=p=0.005, **=p=0.001).

FIG. 4: Spontaneous resorption mechanism cascade as proposed by Kato et al. An inflammatory reaction starts when macrophages are activated upon contact with disc tissue, causing an upregulated TNF expression. TNF induces Vascular Endothelial Growth Factor (VEGF) and MMP's. TNF also induces plasminogen activators (PA) generating plasmin which in turs also activates MMP's. MMP's are then responsible for the degradation of the herniated disc matrix leading to spontaneous resorption.

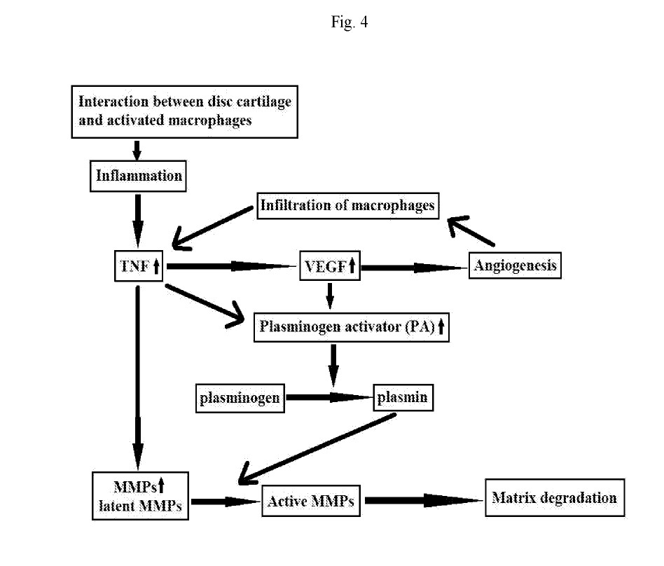

### DESCRIPTION OF THE INVENTION

It has surprisingly been found that inhibitors of CD28-mediated co-stimulation of T-cells are useful in the treatment and prophylaxis of intervertebral disc herniation in mammals. According to the invention, inhibition of CD28-mediated co-stimulation of T-cells prevents formation of disc hernias; as well as causes the disc hernia to decrease in size, thus relieving the nerve root from pressure. This is in sharp contrast to the previous belief that autoimmunity contributes to spontaneous resorption. Further, inhibition of CD28-mediated co-stimulation of T-cells is likely to reduce inflammation and thus cytokines known to be important for the sensitization of the nerve root.

Consequently, in a first aspect the invention provides a composition for use in the treatment or prophylaxis of intervertebral disc herniation in a mammal, such as a human, said composition comprising a modulator of T-cell co-stimulation, such as an inhibitor of T-cell activation. The term “treatment or prophylaxis” as used herein includes (i) preventing the disease, i.e. causing the clinical symptoms of the disease not to develop; (ii) inhibiting the disease, i.e. arresting the development of clinical symptoms; and/or (iii) relieving the disease, i.e. causing the regression, amelioration or elimination of clinical symptoms.

Preferably, the said inhibitor of T-cell activation is a protein, which is capable of binding to CD80/CD86. The term “CD80/CD86” refers to the proteins Cluster of Differentiation 80 (also known as B7-1) and Cluster of Differentiation 86 (also known as B7-2), which work in tandem to prime T-cells. The said proteins are found on antigen-presenting cells (APCs) and provide costimulatory signals necessary for T-cell activation and survival. CD80/CD86 are ligands for two different proteins on the T-cell surface: CD28 (for autoregulation and intercellular association) and CTLA-4 (for attenuation of regulation and cellular disassociation).

More preferably, the said inhibitor of T-cell activation is a protein comprising at least one extracellular domain of CTLA-4 (cytotoxic T-lymphocyte-associated protein 4; also known as CD152 (cluster of differentiation 152)). Specifically, the said protein could comprise one extracellular domain or, preferably, two extracellular domains of CTLA-4. CTLA-4 is a protein receptor, which transmits an inhibitory signal to T cells when bound to CD80 or CD86 on the surface of antigen-presenting cells.

The said protein comprising the extracellular domain of CTLA-4 is preferably a fusion protein comprising the fragment crystallizable (Fc) region of IgG fused to the extracellular domain of CTLA-4. The said protein could be a monomer or, preferably, a dimer of such a fusion protein. The said immunoglobulin G can be selected from any subclass of IgG (IgG1, IgG2, IgG3 and IgG4). Preferably, the said IgG is IgG1. Alternatively, the fusion protein can comprise the Fc region of other immunoglobulins, including IgA1, IgA2, IgD, IgE and IgM.

The said extracellular domain of CTLA-4 is preferably chosen from:
(a) a polypeptide having an amino acid sequence comprising the sequence shown as SEQ ID NO: 3 or SEQ ID NO: 4;
(b) a polypeptide having an amino acid sequence which is at least 90% identical, preferably at least 95%, 96%, 97%, 98% or 99% identical, with the sequence shown as SEQ ID NO: 3 or SEQ ID NO: 4;
(c) a polypeptide having an amino acid sequence consisting essentially of the sequence shown as SEQ ID NO: 3 or SEQ ID NO: 4; or
(d) a polypeptide having an amino acid sequence consisting of the sequence shown as SEQ ID NO: 3 or SEQ ID NO: 4.

Preferably, the said fusion protein is a dimer comprising two identical or non-identical monomers. Each monomer in the said fusion protein is preferably chosen from:
(a) a polypeptide having an amino acid sequence comprising the sequence shown as SEQ ID NO: 1 or SEQ ID NO: 2;
(b) a polypeptide having an amino acid sequence which is at least 90% identical, preferably at least 95%, 96%, 97%, 98% or 99% identical, with the sequence shown as SEQ ID NO: 1 or SEQ ID NO: 2;
(c) a polypeptide having an amino acid sequence consisting essentially of the sequence shown as SEQ ID NO: 1 or SEQ ID NO: 2; or
(d) a polypeptide having an amino acid sequence consisting of the sequence shown as SEQ ID NO: 1 or SEQ ID NO: 2.

The polypeptides shown as SEQ ID NO: 1 and 2 represent monomers of the fusion proteins known as abatacept and belatacept, respectively. The polypeptides shown as SEQ ID NO: 3 and 4 represent the extracellular part of CTLA4 used in abatacept and belatacept, respectively.

Alternatively, the said inhibitor of CD28-mediated co-stimulation of T-cells is a protein comprising affinity and antagonistic properties towards CD28. More specifically, the said protein could comprise a monovalent pegylated Fab′ Ab antagonist of CD28, such as FR104.

Other known modulators and/or inhibitors of CD28-mediated co-stimulation of T-cells include AB103, anti-CD86 Monoclonal Antibody AIDA, BMS931699, CTLA4Ig MEDEXGEN, Debio0615; RG2077, and ShK186. Further, the said inhibitor of CD28-mediated co-stimulation of T-cells could be a protein comprising affinity and antagonistic properties towards CD86, such as ES210 or PG140, or towards CD80, such as IDEC114, KNO18, KNO19, Maxy30, or RhuDex.

In the present context, the term “comprising” should not be understood to be exclusively restricted to the recited amino acid sequences, instead it should be understood by the open-ended meaning “including” or “containing”. Consequently, the term “comprising” does not exclude sequences having additional, unrecited amino acids.

The “identity” of a sequence with a reference sequence refers to the percent of residues that are the same when the two sequences are aligned for maximum correspondence between residues positions. More specifically, the “sequence identity” expressed in percentage is defined as the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Unless indicated otherwise, the comparison window is the entire length of the sequence being referred to. In this context, optimal alignment is the alignment produced by the BLASTP algorithm as implemented online by the US National Center for Biotechnology Information (see The NCBI Handbook [Internet], Chapter 16), with the following input parameters: Word length=3, Matrix=BLOSUM62, Gap cost=11, Gap extension cost=1.

The term “consisting essentially of” is intended to exclude that which materially changes basic and novel characteristics of the invention. Consequently, polypeptides having an amino acid sequence consisting essentially of the sequence shown as SEQ ID NOS: 1 to 4 encompasses polypeptides carrying modifications like substitutions, small deletions, insertions or inversions, which polypeptides nevertheless have substantially the biological activities of the polypeptides shown as SEQ ID NOS: 1 to 4.

It is envisaged that other components of the adaptive immune system could be used according to the invention, either on their own, or in combination with each other or with T-cell inhibitors as defined above. Such components include, but are not limited to, various interleukin inhibitors such as anakinra, tocilizumab, basiliximab, ustekinumab, kanakinumab, sekukinumab, siltuximab; TNF-inhibitors such as etanercept, infliximab, afelimomab, adalimumab, golimumab, cetrolizumabpegol; calcineurin inhibitors such as ciclosporin or tacrolimus; B-cell inhibitors such as rituximab; or other immunomodulating drugs with effects on the adaptive immune system such as immunoglobulin therapy, glucocorticoids, methotrexate, leflunomide, sulfasalazine, azathioprine, cyclophosphamide or doxycycline.

As mentioned above, the composition for use according to the invention is useful in the treatment or prophylaxis of intervertebral disc herniation. The terms “disc herniation” and “disc hernia” are to be understood as known in the art, see e.g. Fardon et al. [50]. Consequently, “herniation” is broadly defined as a localized or focal displacement of disc material beyond the limits of the intervertebral disc space. The presence of disc tissue extending beyond the edges of the ring apophyses, throughout the circumference of the disc, is called “bulging”. Herniated discs may be classified as “protrusion” or “extrusion”, based on the shape of the displaced material. “Protrusion” is present if the greatest distance between the edges of the disc material presenting outside the disc space is less than the distance between the edges of the base of that disc material extending outside the disc space. The base is defined as the width of disc material at the outer margin of the disc space of origin, where disc material displaced beyond the disc space is continuous with the disc material within the disc space. “Extrusion” is present when, in at least one plane, any one distance between the edges of the disc material beyond the disc space is greater than the distance between the edges of the base of the disc material beyond the disc space or when no continuity exists between the disc material beyond the disc space and that within the disc space. The latter form of extrusion is best further specified or subclassified as “sequestration” if the displaced disc material has lost continuity completely with the parent disc. For further information, see reference [50] and drawings therein.

Consequently, the term “treatment or prophylaxis of intervertebral disc herniation” should be understood as comprising at least one, and preferably two, three, four or five, of the following effects:
(i) reduction or prophylaxis of inflammation;
(ii) reduction or prophylaxis of the formation of disc herniations, including protrusions, extrusions and sequestrations; and
(iii) reduction of the size of disc herniations, including protrusions, extrusions and sequestrations;
(iv) reduction or prophylaxis of bulge formation; and
(v) reduction of the size of disc bulges.

In this context the term “reduction” is intended to encompass partial reduction, as well as complete inhibition or elimination of the said conditions.

As mentioned above in the Background Art section, human disc hernias have been found to comprise various types of tissue, including nucleus pulposus, annulus fibrosus, cartilaginous endplate and granulation tissue (scar tissue). The relative amount of granulation tissue has been found to be higher in younger patients. Granulation tissue has also been found to be more present in sequestrations and extrusions as compared to bulges and protrusions. The compositions for use according to the invention are useful in the treatment of disc hernias of all the said forms, and may be particularly useful for the treatment of younger patients with disc extrusions or sequestrations. Consequently, in a preferred aspect the invention provides compositions, as disclosed herein, for use in the treatment or prophylaxis of intervertebral disc herniation in a mammal, wherein the said treatment or prophylaxis comprises reduction or prevention of the formation of granulation tissue, especially wherein the said disc herniation comprises extrusions and/or sequestrations.

Further, the term “intervertebral disc herniation” includes conditions characterized by radiating pain and/or back pain, which conditions are deemed by a physician likely to be caused by intervertebral disc herniation, with or without radiological confirmation with e.g. MRI. Moreover, the intervertebral disc herniation can be located at various locations in the spine, all of which are included in this definition, including cervical, thoracic and lumbar intervertebral disc herniation.

The composition for use according to the invention can be administered to the patient in various ways, such as intravenously, intramuscularly, subcutaneously, intraperitoneally, orally, topically, rectally, etc. Further, the composition can be administered by a slow-release device, such as a miniosmotic pump implanted into the subject's body.

For instance, when the composition comprises a fusion protein such as abatacept, belatacept, ASP2408 or XPro9523, including combinations thereof, it could be administered in accordance with the methods known in the art for the said compounds. For example, the fusion protein could be administered as intravenous infusion in a dose of 0.1-20 mg/kg body weight, such as 0.5-20, 1-20, 5-20, or 5-15 mg/kg body weight, or more preferably around 10 mg/kg body weight. Alternatively, fusion protein could be administered in a dose of 500 mg for patients below 60 kg body weight; 750 mg for patients 60 to 100 kg body weight; or 1000 mg for patients above 100 kg body weight. Following the initial intravenous administration, an intravenous infusion could be given at 2 and 4 weeks after the first infusion and every 4 weeks thereafter.

Alternatively, the composition could be administered by subcutaneous injection, e.g. in a dose of 125 mg fusion protein once weekly. Such treatment may be initiated with or without an intravenous loading dose. For patients initiating therapy with an intravenous loading dose, treatment could be initiated with a single intravenous infusion, followed by the first 125 mg subcutaneous injection administered within a day of the intravenous infusion.

Further, the composition could be formulated such that it may be administered in a therapeutically effective amount by local injection to an intervertebral disc.

In a further aspect, the invention provides a method for the treatment or prophylaxis of intervertebral disc herniation, comprising administering to a mammal, such as a human, in need thereof a therapeutically effective dose of an inhibitor of T-cell activation. Effects and features of this second aspect of the present invention are analogous to those described above in relation to the first aspect of the present invention. In particular, the said inhibitor of T-cell activation is preferably a fusion protein, such as abatacept or belatacept, comprising the Fc region of immunoglobulin IgG1, fused to the extracellular domain of CTLA-4.

### EXAMPLES

### Example 1: Pilot Study, Treatment with Abatacept

20 female Sprague-Dawley rats weighing approximately 225 g were evenly divided into two groups: disc puncture (DP, n=10) and disc puncture+abatacept (DP+abatacept, n=10). The rats were housed with free access to food and water in environmental-enriched cages. All rats underwent the same surgical procedure as described below.

Anesthesia was induced and maintained by the inhalation of Isoflurane. A single dose of 0.05 mg/kg buprenorphine (Temgesic®) was administered subcutaneously before the procedure for per- and postoperative analgesia.

Following induction of anesthesia, a skin incision of approximately 6 cm was made in the midline over the spinous processes of the lumbar and caudal spine. The spinal muscles on the left side of the lumbar spine were dissected to expose the left L4/L5 facet joint. The left L4/L5 facet joint was removed to expose the underlying dural sac, L4 nerve root and L4/L5 intervertebral disc. The disc was then punctured using a 23 g needle. A small amount of air (0.2-0.3 ml) was injected into the disc to facilitate leakage of nucleus pulposus. The spinal muscles and the thoracolumbar fascia were then sutured and the skin closed with metal clips.

For the DP group, no additional treatment was given. For the DP+abatacept group, 10 mg/kg abatacept was administered intraperitoneally immediately before surgery. This was repeated 7 and 14 days after surgery.

To evaluate the effects on the morphology of the hernia-like nodule a macroscopic analysis was performed. 21 days after surgery anesthesia was again induced by inhalation of isoflurane followed by euthanization by incision of the heart, causing the rat to quickly bleed to death. The lumbar spine was then dissected by removing the spinal muscles and the vertebral arches of L4 and L5, exposing the L4/L5 intervertebral disc. A macroscopic analysis was performed through the surgical microscope by an experienced researcher. A description of the different types of data collected can be found in Table 1.

The data from the macroscopic analysis was considered ordinal. To test for statistical significance between the DP and DP+abatacept groups, Mann-Whitney U-tests were performed. Significance was defined as p≤0.05.

All rats survived the surgery and displayed good general condition throughout the study. In the DP group, 8 out of 10 rats displayed clear or pronounced disc nodules (Table 2). In the DP+abatacept group, no clear or pronounced nodules were found. The difference in nodule size was significant between the two groups (p=0.000). There was also significantly less inflammation observed in the DP+abatacept group (p=0.005). Tendencies of less osteophytes were also noted, but this difference was not significant (p=0.143).

### Example 2: Longitudinal Assessment of Nodule Size, Treatment with Abatacept

The purpose of this study was to assess 1) how the nodule formed after disc puncture in the rat varies in size over time, 2) if the formation of the nodule can be inhibited by administration of the T-cell inhibitor abatacept, and 3) if the size of existing nodules can be reduced by administration of the T-cell inhibitor abatacept.

24 female Sprague-Dawley rats (n=24) weighing approximately 225 g were evenly divided into three groups: Control group (no treatment, n=8); Treatment start day 0 (n=8); and Treatment start day 14 (n=8). The rats were housed with free access to food and water in environmental-enriched cages. All rats underwent the same surgical procedure as described below.

Anesthesia was induced and maintained by the inhalation of Isoflurane. A single dose of 0.05 mg/kg buprenorphine (Temgesic®) was administered subcutaneously before the procedure for per- and postoperative analgesia.

Following induction of anesthesia, a skin incision of approximately 6 cm was made in the midline over the spinous processes of the lumbar and caudal spine. The spinal muscles on the left side of the lumbar spine were dissected to expose the left L5/L6 facet joint. The left L5/L6 facet joint was removed to expose the underlying dural sac, L5 nerve root and L5/L6 intervertebral disc. The disc was punctured using a 23 g needle. A small amount of air (0.2-0.3 ml) was injected into the disc to facilitate leakage of nucleus pulposus. The spinal muscles and the thoracolumbar fascia were then sutured and the skin closed with metal clips. The researcher performing the surgery was blinded to treatment groups.

All rats received an intraperitoneal injection immediately before surgery and subsequently once a week throughout the study period. For the different groups, the compound administered at the different time periods were as shown in Table 3.

For dosage calculations, a body weight of 225 g was assumed for all rats throughout the study period. When abatacept was not administered, saline was administered for blinding purposes. The rats were shortly anesthetized by inhalation of isoflurane before each injection to allow for a safe administration of the compound and to minimize pain and stress to the animal.

To assess the size of the disc nodules following disc puncture, MRI images were obtained with a 7T MRI system (Bruker Bio Spec®) week 1, 2, 4 and 8 after surgery. The rats were anesthetized by inhalation of isoflurane throughout the MRI measurements. During the measurements, breathing frequency was monitored and the body temperature maintained by placing the rat on a heated pad.

A sagittal T2-weighted 3D RARE sequence with isotropic voxels of 150 μm was used. The volume of the nodule was measured by manually defining a region of interest (ROI) of the disc aggrandizement dorsal to a longitudinal line between the most dorsal aspects of the adjacent vertebrae on each image where the disc could be identified. The number of voxels within the ROI was assessed three times for each image. The average number of voxels for each sagittal section of the disc were added and then multiplied by the voxel volume (0.003375 mm³), resulting in the total nodule size expressed in mm³. In addition to this, a subjective assessment of the nodule size was also performed by the observer for each MM. The researcher collecting the data was blinded to treatment groups.

Upon termination of the study, a macroscopic analysis was performed in addition to the MRI measurements. After the final MRI measurement 8 weeks after surgery the rats were euthanized by incision of the heart while still anesthetized by inhalation of isoflurane, causing the rat to quickly bleed to death. The lumbar spine was dissected by removing the spinal muscles and the vertebral arches of L5 and L6, exposing the L5/L6 intervertebral disc. A macroscopic assessment of the nodule size was performed through the surgical microscope. The assessments of the nodule size were performed according to the description in Table 1. The observer was not blinded to treatment groups for logistic reasons.

All rats survived the surgery and displayed good general condition throughout the study. The data from the macroscopic analysis and the subjective assessment from the MRI were considered ordinal. The data from the volume measurement on the MM was considered continuous Mann-Whitney U-tests were performed, and statistical significance defined as p≤0.05.

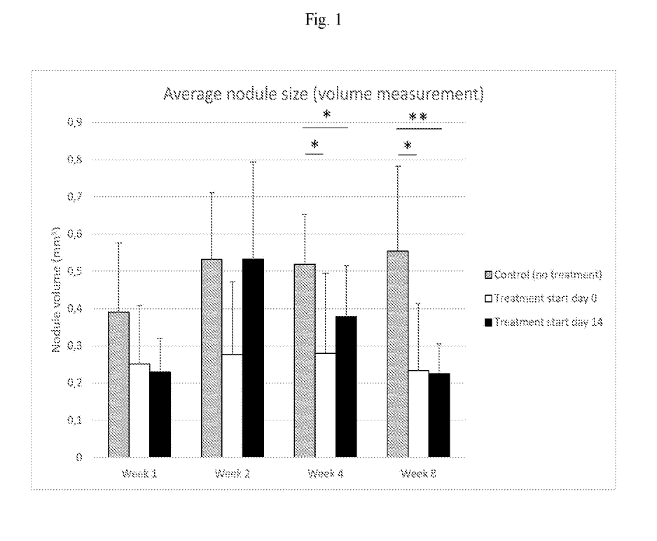

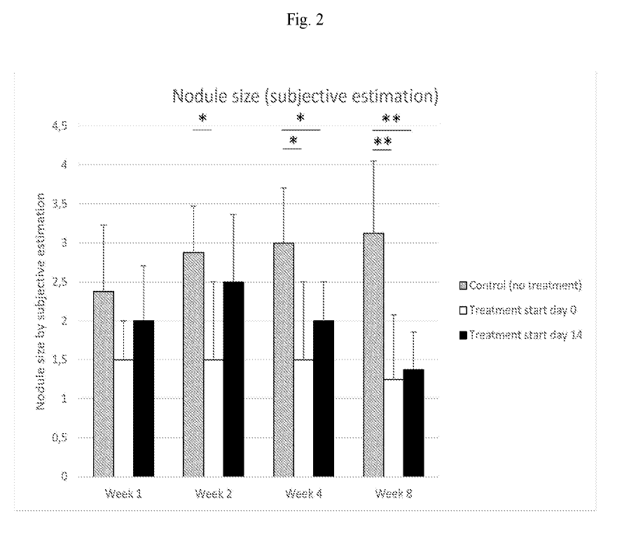

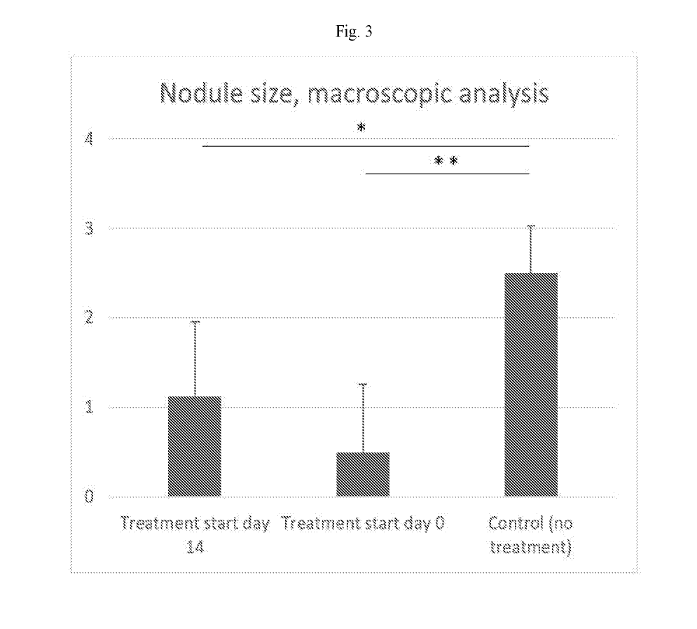

The results from the MM measurements can be found in FIG. 1 and FIG. 2. The results from the macroscopic analysis can be found in FIG. 3. The nodule was found to be significantly smaller in both treatment groups as compared to control by all types of assessments on week 4 and 8. In addition, a significant difference in nodule size as determined by subjective assessment was noted between “Control (no treatment)” and “Treatment start day 0” on week 2.

To conclude, the results of this study confirmed that administration of abatacept inhibited the formation of the hernia-like nodule when treatment started immediately before disc puncture. Also, when treatment with abatacept was initiated two weeks after disc puncture, the hernia-like nodules that had formed during the two weeks became significantly smaller compared to the control group, indicating that abatacept can also cause shrinkage of the hernia-like nodule after it has formed. These results contradict the generally accepted hypothesis that autoimmunity and inflammation are beneficial for the resorption of hernias, but rather suggest that specific inhibition of the immune system, more specifically T-cells, can cause resorption and thus shrinkage of hernias.

### Example 3: Longitudinal Assessment of Nodule Size and Immunohistochemistry, Treatment with Specific Inhibitor of CD28-Mediated Co-Stimulation of T-Cells

The purpose of this study is 1) to reproduce previous results indicating that treatment with a specific inhibitor of CD28-mediated co-stimulation of T-cells can be used to induce and/or expedite resorption of disc hernias in the modified animal model described in Example 3, and 2) to make a more detailed assessment of specific morphological changes in the hernia-like nodules caused by treatment with a specific inhibitor of CD28-mediated co-stimulation of T-cells.

48 female Sprague-Dawley rats weighing approximately 225 g are evenly divided into three groups: Control group (Control, n=16); low dose treatment (T-L n=16); high dose treatment (T-H, n=16). The rats are housed with free access to food and water in environmental-enriched cages. All rats undergo the same surgical procedure as described below. The researcher performing the surgery is blinded to treatment groups.

Anesthesia is induced and maintained by the inhalation of Isoflurane. A single dose of 0.05 mg/kg buprenorphine (Temgesic®) is administered subcutaneously before the procedure for per- and postoperative analgesia.

Following induction of anesthesia, a skin incision of approximately 5 cm is made in the midline over the spinous processes of the lumbar and caudal spine. An incision is made in the thoracolumbar fascia from level L3-L6. The spinal muscles on both sides on level L4/5 are dissected as well as the supra- and interspinous ligaments to expose the laminae and the interlaminar space at L4/5. A small laminotomy is performed on the caudal aspect of L4 using a surgical micro drill. A 23 g needle is then used to carefully incise the exposed dural sac. The needle is introduced into the cauda equina and the underlying L4/5 disc identified by probing with the needle. The disc is punctured and a small amount of air (0.2 ml) is injected into the disc to facilitate leakage of nucleus pulposus. The needle is then removed, the thoracolumbar fascia sutured and the skin closed with metal clips.

Therapeutic compounds are administered by intraperitoneal injections once a week.

To assess the size of the disc nodules following disc puncture, MRI images are obtained with a 7T MRI system (Bruker BioSpec®). Measurements are performed week 24 and 8 after surgery. The rats are anesthetized by inhalation of isoflurane throughout the MRI measurements. During the measurements, breathing frequency is monitored and the body temperature maintained by placing the rat on a heated pad.

A sagittal T2-weighted 3D RARE sequence with isotropic voxels of 150 μm is used. The volume of the nodule is measured by manually defining a region of interest (ROI) of the disc aggrandizement dorsal to a longitudinal line between the most dorsal aspects of the adjacent vertebrae on each image where the disc can be identified. The number of voxels within the ROI is assessed three times for each image. The average number of voxels for each sagittal section of the disc are added and then multiplied by the voxel volume (0.003375 mm³), resulting in the total nodule size expressed in mm³. In addition to this, a subjective assessment of the nodule size is also performed by the observer for each MM. The researcher collecting the data is blinded to treatment groups.

Upon termination of the study, a macroscopic analysis is performed in addition to the MRI measurements. After the final MRI measurement 8 weeks after surgery the rats are euthanized by incision of the heart while still anesthetized by inhalation of isoflurane, causing the rat to quickly bleed to death. The lumbar spine is dissected by removing the spinal muscles and the vertebral arches of L3 through L6, exposing the underlying intervertebral disc. A macroscopic assessment of the nodule size is performed through the surgical microscope. The assessments of the nodule size were performed according to the description in Table 1.

Some of the animals from all groups are terminated at various time points previous to week 8 to allow for harvest of the punctured intervertebral discs for assessments with histology and immunohistochemistry.

The results from this study support that targeted inhibition of T cell activation, more specifically inhibition of CD28-mediated co-stimulation, can be used to treat disc herniation by inducing and/or expediting resorption of the hernia as well as reducing inflammation in the hernia.

### Example 4: Treatment of Disc Herniation with an Inhibitor of CD28-Mediated Co-Stimulation of T-Cells, Patient Scenario

This example is a fictional case report meant to illustrate how treatment with an inhibitor of CD28-mediated co-stimulation of T-cells is intended to be used and hypothesized to work in the treatment of disc herniation in a clinical setting.

A 40-year-old woman presents to the primary care facility with a 6 week history of low back pain and sciatic pain in her left leg. She has experienced back pain on occasion before but is otherwise healthy without any medication. She has now tried physiotherapy with limited symptom relief and is still unable to work due to the severity of her symptoms. The primary care physician performs a physical examination and finds the patient has reduced sensory function in her left leg corresponding to the dermatome of 51. The left Achilles reflex is weaker than the right and she has reduced strength in plantar flexion of the left ankle. A left-sided positive Laségue's sign is noted with pain distribution corresponding to the 51 nerve root.

The primary care physician suspects a lumbar disc herniation with radiculopathy of the left 51 nerve root. A referral is written for MRI of the lumbar spine to confirm the diagnosis. The patient is also given a prescription for acetaminophen and a non-steroidal anti-inflammatory drug for symptom relief.

Four weeks later, the patient has undergone MRI and is back at the primary care facility for reassessment. She has not experienced any improvement in her symptoms and uses the prescribed analgesics daily. MRI has confirmed a L5/S1 left-sided disc extrusion with deformation of the left 51 nerve root. A referral is now sent to the spine clinic where the patient is accepted for a new outpatient reassessment in three weeks.

At the spine clinic, the patient confirms the absence of improvement the past two months. Through physical assessment and assessment of MR images the physician at the spine clinic concludes that the patient has a L5/S1 disc extrusion with symptoms corresponding to this finding. Since there is no spontaneous improvement in symptoms and after ruling out contraindications, the patient is offered and accepts treatment with a drug that inhibits the CD28-mediated co-stimulation of T-cells. A nurse at the spine clinic shows the patient how to self-administer the drug, for example subcutaneously.

The patient picks up the drug from the local pharmacy and commence treatment at home by administering the drug according to instruction, for example once every one or two weeks. 2-3 weeks after starting treatment the patient starts experiencing improvements in her symptoms, and after 6-8 weeks the symptoms are vastly improved. After a total treatment time of for example 12 weeks, treatment is ended.

On follow-up MRI 6 months after starting treatment there is still a visible extrusion but it has markedly decrease in size and the adjacent nerve root is no longer affected. The patient is still free from symptoms.

TABLE 1
Variables collected during macroscopic analysis.

| | | |
|---|---|---|
| Inflammation | Degree of hyperemia | 0 = No hyperemia |
| | of the disc/−nodule | (+) = Slight hyperemia |
| | | + = Clear hyperemia |
| | | ++ = Pronounced hyperemia |
| Healing | Healing of puncture | 0 = No healing |
| | site | (+) = Gel-like healing |
| | | + = Elastic |
| | | N/A = Not applicable |
| Nodule size | Size of lesion over | 0 = No nodule |
| | the puncture site | (+) = Small nodule |
| | | + = Clear nodule |
| | | ++ = Pronounced nodule |
| Nodule | Consistency of | (+) = Gel-like healing |
| consistency | nodule as determined | + = Elastic |
| | by probing | ++ = Hard |
| | | N/A = Not applicable |
| Osteophytes | Osteophytes on | 0 = No osteophytes |
| | adjacent vertebrae | (+) = Tendency of osteophytes |
| | | + = Clear osteophytes |
| | | ++ = Pronounced osteophytes |
| Infection | Pus noted during | 0 = Not present |
| | dissection | + = Present |

TABLE 2
Results from macroscopic analysis.
Variables as defined in Table 1.

| | | | | | |
|---|---|---|---|---|---|
| | | | Nodule | Nodule | |
| Rat | Inflammation | Healing | size | consistency | Osteophytes |

DP (Control)

| | | | | | |
|---|---|---|---|---|---|
| 1 | (+) | + | ++ | (+) | 0 |
| 2 | 0 | + | 0 | − | (+) |
| 3 | (+) | + | (+) | (+) | (+) |
| 4 | (+) | + | ++ | (+) | + |
| 5 | + | + | ++ | (+) | 0 |
| 6 | (+) | + | + | (+) | (+) |
| 7 | (+) | + | + | (+) | + |
| 8 | (+) | + | + | + | + |
| 9 | (+) | + | ++ | + | ++ |
| 10 | (+) | + | ++ | (+) | 0 |

DP + abatacept

| | | | | | |
|---|---|---|---|---|---|
| 11 | 0 | 0 | 0 | − | 0 |
| 12 | 0 | 0 | 0 | − | (+) |
| 13 | 0 | + | 0 | − | (+) |
| 14 | 0 | + | 0 | − | (+) |
| 15 | (+) | + | 0 | − | 0 |
| 16 | (+) | + | (+) | (+) | (+) |
| 17 | 0 | + | 0 | − | 0 |
| 18 | 0 | + | (+) | (+) | (+) |
| 19 | 0 | + | 0 | − | 0 |
| 20 | 0 | + | 0 | − | 0 |

TABLE 3
Outline of study for assessment of nodule size.

| | 0 | 1 | 2 | 3 | 4 | 5 | 6 | 7 |
|---|---|---|---|---|---|---|---|---|
| Group | Week | | | | | | | |
| Control | Saline | Saline | Saline | Saline | Saline | Saline | Saline | Saline |
| Treatm. | Abat. | Abat. | Abat. | Abat. | Abat. | Abat. | Abat. | Abat. |
| start day 0 | 10 mg/kg | 10 mg/kg | 10 mg/kg | 10 mg/kg | 10 mg/kg | 10 mg/kg | 10 mg/kg | 10 mg/kg |
| Treatm. | Saline | Saline | Abat. | Abat. | Abat. | Abat. | Abat. | Abat. |
| start day | | | 10 mg/kg | 10 mg/kg | 10 mg/kg | 10 mg/kg | 10 mg/kg | 10 mg/kg |
| 14 | | | | | | | | |

Abat. = Abatacept.

### REFERENCES

1. Brisby, H., Pathology and possible mechanisms of nervous system response to disc degeneration. J Bone Joint Surg Am, 2006. 88 Suppl 2: p. 68-71.
2. Kuslich, S. D., C. L. Ulstrom, and C. J. Michael, The tissue origin of low back pain and sciatica: a report of pain response to tissue stimulation during operations on the lumbar spine using local anesthesia. Orthop Clin North Am, 1991. 22(2): p. 181-7.
3. Mulleman, D., et al., Pathophysiology of disk-related sciatica. I.—Evidence supporting a chemical component. Joint Bone Spine, 2006. 73(2): p. 151-8.
4. Olmarker, K., B. Rydevik, and C. Nordborg, Autologous nucleus pulposus induces neurophysiologic and histologic changes in porcine cauda equina nerve roots. Spine (Phila Pa. 1976), 1993. 18(11): p. 1425-32.
5. Olmarker, K., et al., Ultrastructural changes in spinal nerve roots induced by autologous nucleus pulposus. Spine (Phila Pa. 1976), 1996. 21(4): p. 411-4.
6. Byrod, G., et al., Early effects of nucleus pulposus application on spinal nerve root morphology and function. Eur Spine J, 1998. 7(6): p. 445-9.
7. Olmarker, K. and K. Larsson, Tumor necrosis factor alpha and nucleus-pulposus-induced nerve root injury. Spine (Phila Pa. 1976), 1998. 23(23): p. 2538-44.
8. Risbud, M. V. and I. M. Shapiro, Role of cytokines in intervertebral disc degeneration: pain and disc content. Nat Rev Rheumatol, 2014. 10(1): p. 44-56.
9. Kadow, T., et al., Molecular basis of intervertebral disc degeneration and herniations: what are the important translational questions? Clin Orthop Relat Res, 2015. 473(6): p. 1903-12.
10. Korhonen, T., et al., The treatment of disc-herniation-induced sciatica with infliximab: one-year follow-up results of FIRST II, a randomized controlled trial. Spine (Phila Pa. 1976), 2006. 31(24): p. 2759-66.
11. Freeman, B. J., et al., Randomized, double-blind, placebo-controlled, trial of transforaminal epidural etanercept for the treatment of symptomatic lumbar disc herniation. Spine (Phila Pa. 1976), 2013. 38(23): p. 1986-94.
12. de Souza Grava, A. L., L. F. Ferrari, and H. L. Defino, Cytokine inhibition and time-related influence of inflammatory stimuli on the hyperalgesia induced by the nucleus pulposus. Eur Spine J, 2012. 21(3): p. 537-45.
13. Cuellar, J. M., et al., Cytokine expression in the epidural space: a model of noncompressive disc herniation-induced inflammation. Spine (Phila Pa. 1976), 2013. 38(1): p. 17-23.
14. Takahashi, H., et al., Inflammatory cytokines in the herniated disc of the lumbar spine. Spine (Phila Pa. 1976), 1996. 21(2): p. 218-24.
15. Andrade, P., et al., Elevated IL-1beta and IL-6 levels in lumbar herniated discs in patients with sciatic pain. Eur Spine J, 2013. 22(4): p. 714-20.
16. Pennington, J. B., R. F. McCarron, and G. S. Laros, Identification of IgG in the canine intervertebral disc. Spine (Phila Pa. 1976), 1988. 13(8): p. 909-12.
17. Satoh, K., et al., Presence and distribution of antigen-antibody complexes in the herniated nucleus pulposus. Spine (Phila Pa. 1976), 1999. 24(19): p. 1980-4.
18. Capossela, S., et al., Degenerated human intervertebral discs contain autoantibodies against extracellular matrix proteins. Eur Cell Mater, 2014. 27: p. 251-63; discussion 263.
19. Geiss, A., et al., Autologous nucleus pulposus primes T cells to develop into interleukin-4-producing effector cells: an experimental study on the autoimmune properties of nucleus pulposus. J Orthop Res, 2009. 27(1): p. 97-103.
20. Pedersen, L. M., et al., Serum levels of the pro-inflammatory interleukins 6 (IL-6) and-8 (IL-8) in patients with lumbar radicular pain due to disc herniation: A 12-month prospective study. Brain Behav Immun, 2015.
21. Yasuma, T., K. Arai, and Y. Yamauchi, The histology of lumbar intervertebral disc herniation. The significance of small blood vessels in the extruded tissue. Spine (Phila Pa. 1976), 1993. 18(13): p. 1761-5.
22. Kokubo, Y., et al., Herniated and spondylotic intervertebral discs of the human cervical spine: histological and immunohistological findings in 500 en bloc surgical samples. Laboratory investigation. J Neurosurg Spine, 2008. 9(3): p. 285-95.
23. Matsui, Y., et al., The involvement of matrix metalloproteinases and inflammation in lumbar disc herniation. Spine (Phila Pa. 1976), 1998. 23(8): p. 863-8; discussion 868-9.
24. Matveeva, N., et al., Histological composition of lumbar disc herniations related to the type of herniation and to the age. Bratisl Lek Listy, 2012. 113(12): p. 712-7.
25. Lee, J. Y., et al., Histological study of lumbar intervertebral disc herniation in adolescents. Acta Neurochir (Wien), 2000. 142(10): p. 1107-10.
26. Winkler, D., et al., Does histology predict the clinical outcome after lumbar intervertebral disc herniation: no. Med Hypotheses, 2013. 80(3): p. 215-9.
27. Jensen, T. S., et al., Natural course of disc morphology in patients with sciatica: an MRI study using a standardized qualitative classification system. Spine (Phila Pa. 1976), 2006. 31(14): p. 1605-12; discussion 1613.
28. Takada, E., M. Takahashi, and K. Shimada, Natural history of lumbar disc hernia with radicular leg pain: Spontaneous MRI changes of the herniated mass and correlation with clinical outcome. J Orthop Surg (Hong Kong), 2001. 9(1): p. 1-7.
29. Splendiani, A., et al., Spontaneous resolution of lumbar disk herniation: predictive signs for prognostic evaluation. Neuroradiology, 2004. 46(11): p. 916-22.
30. Henmi, T., et al., Natural history of extruded lumbar intervertebral disc herniation. J Med Invest, 2002. 49(1-2): p. 40-3.
31. Genevay, S., et al., Influence of cytokine inhibitors on concentration and activity of MMP-1 and MMP-3 in disc herniation. Arthritis Res Ther, 2009. 11(6): p. R169.
32. Seo, J. Y., et al., Three-dimensional analysis of volumetric changes in herniated discs of the lumbar spine: does spontaneous resorption of herniated discs always occur? Eur Spine J, 2016. 25(5): p. 1393-402.
33. Chiu, C. C., et al., The probability of spontaneous regression of lumbar herniated disc: a systematic review. Clin Rehabil, 2015. 29(2): p. 184-95.
34. Elliott, D. M., et al., The effect of relative needle diameter in puncture and sham injection animal models of degeneration. Spine (Phila Pa. 1976), 2008. 33(6): p. 588-96.
35. Keorochana, G., et al., The effect of needle size inducing degeneration in the rat caudal disc: evaluation using radiograph, magnetic resonance imaging, histology, and immunohistochemistry. Spine J, 2010. 10(11): p. 1014-23.
36. Zhang, H., et al., Time course investigation of intervertebral disc degeneration produced by needle-stab injury of the rat caudal spine: laboratory investigation. J Neurosurg Spine, 2011. 15(4): p. 404-13.
37. Grunert, P., et al., Assessment of intervertebral disc degeneration based on quantitative magnetic resonance imaging analysis: an in vivo study. Spine (Phila Pa. 1976), 2014. 39(6): p. E369-78.
38. Olmarker, K., Puncture of a disc and application of nucleus pulposus induces disc herniation-like changes and osteophytes. An experimental study in rats. Open Orthop J, 2011.5: p. 154-9.
39. Carragee, E. J., et al., 2009 ISSLS Prize Winner: Does discography cause accelerated progression of degeneration changes in the lumbar disc: a ten-year matched cohort study. Spine (Phila Pa. 1976), 2009. 34(21): p. 2338-45.
40. Olmarker, K., Formation Of Nucleus Pulposus-Induced Disc Hernia-Like Nodules On The Disc Surface Is Not Induced By TNF. The Internet Journal of Spine Surgery, 2009. 5(2): p. 1-5.
41. Smith-Garvin, J. E., G. A. Koretzky, and M. S. Jordan, T cell activation. Annu Rev Immunol, 2009. 27: p. 591-619.
42. Sansom, D. M., CD28, CTLA-4 and their ligands: who does what and to whom? Immunology, 2000. 101(2): p. 169-77.
43. Oshima, S., et al., Immunosuppressive effect of ASP2408, a novel CD86-selective variant of CTLA4-Ig, in rats and cynomolgus monkeys. Int Immunopharmacol, 2016. 40: p. 310-317.
44. Oshima, S., et al., ASP2408 and ASP2409, novel CTLA4-Ig variants with CD86-selective ligand binding activity and improved immunosuppressive potency, created by directed evolution. Protein Eng Des Sel, 2016. 29(5): p. 159-67.
45. Zhu, T., et al., Pharmacokinetics, Pharmacodynamics, Safety, and Tolerability of ASP2408, a Potent Selective T-Cell Costimulation Modulator After Single and Multiple Ascending Doses in Healthy Volunteers and RA Patients. Clin Pharmacol Drug Dev, 2016. 5(5): p. 408-25.
46. Bernett, M. J., et al., Immune suppression in cynomolgus monkeys by XPro9523: an improved CTLA4-Ig fusion with enhanced binding to CD80, CD86 and neonatal Fc receptor FcRn. MAbs, 2013. 5(3): p. 384-96.
47. Poirier, N., et al., Preclinical efficacy and immunological safety of FR104, an antagonist anti-CD28 monovalent Fab′ antibody. Am J Transplant, 2012. 12(10): p. 2630-40.
48. Poirier, N., et al., FR104, an antagonist anti-CD28 monovalent fab′ antibody, prevents alloimmunization and allows calcineurin inhibitor minimization in nonhuman primate renal allograft. Am J Transplant, 2015. 15(1): p. 88-100.
49. Poirier, N., et al., First-in-Human Study in Healthy Subjects with FR104, a Pegylated Monoclonal Antibody Fragment Antagonist of CD28. J Immunol, 2016. 197(12): p. 4593-4602.
50. Fardon, D. F., et al., Lumbar disc nomenclature: version 2.0: Recommendations of the combined task forces of the North American Spine Society, the American Society of Spine Radiology and the American Society of Neuroradiology. Spine J, 2014. 14(11): p. 2525-45.

1-18. (canceled)

19. A method for the treatment or prophylaxis of intervertebral disc herniation, comprising administering to a mammal in need thereof a therapeutically effective dose of an inhibitor of CD28-mediated co-stimulation of T-cells, which inhibitor is a protein comprising at least one extracellular domain of CTLA-4; wherein the treatment or prophylaxis comprises (a) reduction or prevention of disc hernia formation; (b) reduction of disc hernia size, or both.

20. The method according to claim 19 wherein the inhibitor of CD28-mediated co-stimulation of T-cells is a protein which is capable of binding to CD80/CD86.

21. (canceled)

22. The method according to claim 19 wherein the protein is a fusion protein comprising the Fc region of immunoglobulin G (IgG) fused to the at least one extracellular domain of CTLA-4.

23. The method according to claim 19 wherein the extracellular domain of CTLA-4 is chosen from:
(a) a polypeptide having an amino acid sequence comprising the sequence shown as SEQ ID NO: 3 or SEQ ID NO: 4;
(b) a polypeptide having an amino acid sequence which is at least 90% identical with the sequence shown as SEQ ID NO: 3 or SEQ ID NO: 4;
(c) a polypeptide having an amino acid sequence consisting essentially of the sequence shown as SEQ ID NO: 3 or SEQ ID NO: 4; or
(d) a polypeptide having an amino acid sequence consisting of SEQ ID NO: 3 or SEQ ID NO: 4.

24. The method according to claim 22 wherein the fusion protein is a dimer comprising two monomers chosen from:
(a) a polypeptide having an amino acid sequence comprising the sequence shown as SEQ ID NO: 1 or SEQ ID NO: 2;
(b) a polypeptide having an amino acid sequence which is at least 90% identical with the sequence shown as SEQ ID NO: 1 or SEQ ID NO: 2;
(c) a polypeptide having an amino acid sequence consisting essentially of the sequence shown as SEQ ID NO: 1 or SEQ ID NO: 2; or
(d) a polypeptide having an amino acid sequence consisting of SEQ ID NO: 1 or SEQ ID NO: 2.

25-27. (canceled)

28. The method according to claim 19, wherein the treatment or prophylaxis of intervertebral disc herniation comprises reduction of inflammation.

29-30. (canceled)

31. The method according to claim 19, wherein the treatment or prophylaxis of intervertebral disc herniation comprises reduction or prevention of the formation of granulation tissue.

32. The method according to claim 19, wherein the intervertebral disc herniation comprises extrusions, sequestrations, or both.

33. The method according to claim 19, wherein the composition is administered intravenously or subcutaneously.

34. The method according to claim 19, wherein the inhibitor of CD28-mediated co-stimulation of T-cells is administered in a dose of between 0.1-20 mg/kg body weight.

35. The method according to claim 19, wherein the inhibitor of CD28-mediated co-stimulation of T-cells is administered by subcutaneous injection once weekly in a dose of 125 mg.

36. The method according to claim 19, wherein the mammal is a human.


SEQUENCE LISTING (4 sequences)

SEQ ID NO: 1 - protein, 357 aa, Artificial Sequence [sequence omitted]

SEQ ID NO: 2 - protein, 357 aa, Artificial Sequence
  L104EA29Y-Ig (Belatacept)
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 124 aa, artificial [sequence omitted]

SEQ ID NO: 4 - protein, 124 aa, artificial
  CTLA4 domain variant
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD